(19) United States
(12) Patent Application Publication
(10) Pub. No.: US 2016/0113800 A1
(43) Pub. Date: Apr. 28, 2016

(54) LONG HOOD RETRIEVAL DEVICE

(71) Applicant: GI Dynamics, Inc., Lexington, MA (US)

(72) Inventors: Andres Chamorro, III, Ashland, MA (US); David A. Melanson, Hudson, NH (US); Barry Maxwell, Spencer, MA (US); Ian K. Parker, Bristol, RI (US)

(21) Appl. No.: 14/918,823

(22) Filed: Oct. 21, 2015

Related U.S. Application Data
(60) Provisional application No. 62/067,130, filed on Oct. 22, 2014.

(51) Int. Cl. A61F 5/00

(52) U.S. Cl. CPC A61F 5/0089; A61F 5/0076

(57) ABSTRACT

An endoscope hood device for moving a gastrointestinal implant device within a natural lumen of a gastrointestinal tract. The endoscope hood device comprises an atraumatic flared retrieval hood configured to attach to and fit over a distal end of an endoscope. The atraumatic flared retrieval hood comprises a length configured to cover a plurality of protrusions on a surface of the gastrointestinal implant device, the plurality of protrusions comprising both proximal facing protrusions and distal facing protrusions of the gastrointestinal implant device. The atraumatic flared retrieval hood comprises a diameter configured to fit within a human esophagus without damage to the esophagus, and comprises a material of a bend radius configured to move slidably against the esophagus without damage to the esophagus.

### RELATED APPLICATION

This application claims the benefit of U.S. Provisional Application No. 62/067,130, filed on Oct. 22, 2014, the entire teachings of which application are incorporated herein by reference.

### BACKGROUND OF THE INVENTION

Endoscope retrieval and repositioning devices have been successfully used for removing and repositioning gastrointestinal implant devices within the gastrointestinal tract. However, there is an ongoing need for endoscope devices that increase safety and ease of use.

### SUMMARY OF THE INVENTION

In accordance with an embodiment of the invention, there is provided an endoscope hood device for moving a gastrointestinal implant device within a natural lumen of a gastrointestinal tract. The endoscope hood device comprises an atraumatic flared retrieval hood that is configured to attach to and fit over a distal end of an endoscope, and that comprises a length configured to cover a plurality of protrusions on a surface of the gastrointestinal implant device, including both proximal facing protrusions and distal facing protrusions of the gastrointestinal implant device. The atraumatic flared retrieval hood comprises a diameter configured to fit within a human esophagus without damage to the esophagus, and comprises a material of a bend radius configured to move slidably against the esophagus without damage to the esophagus.

In an embodiment according to the invention, there is provided an endoscope hood device for moving a gastrointestinal implant device within a natural lumen of a gastrointestinal tract, the device comprising a collapsible, atraumatic flared retrieval hood configured to attach to and fit over a distal end of an endoscope. The hood comprises a length of between about 18 mm and about 36 mm from the distal end of the endoscope to a distal end of the atraumatic flared retrieval hood. The atraumatic flared retrieval hood comprises a largest diameter of between about 10 mm and about 20 mm; a material of a durometer between about Shore 70A and about Shore 95A; and a material of a bend radius of at least about 0.055 inches, the bend radius being measured at a bend of the atraumatic flared retrieval hood when the atraumatic flared retrieval hood is in a flattened configuration.

In further, related embodiments, the bend radius of the atraumatic flared retrieval hood may be at least about 0.060 inches. The bend radius may be measured at a distal tip of the atraumatic flared retrieval hood. The atraumatic flared retrieval hood may comprise at least one of a urethane, an elastomer, and a silicone, such as a transparent urethane.

In another embodiment according to the invention there is provided a retrieval device comprising any of the endoscope hood devices taught herein. The retrieval device may further comprise the endoscope, the endoscope defining a lumen and providing an outer tube of the retrieval device. The retrieval device may further comprise an inner tube defining a lumen and adapted for insertion into the endoscope; an elongated member having a proximal end and a distal end, the elongated member slidably disposed within the lumen of the inner tube; and a grasper disposed at the distal end of the elongated member, adapted to engage the gastrointestinal implant device to collapse at least a portion of the gastrointestinal implant device when operated. The retrieval device may further comprise the gastrointestinal implant device, wherein the gastrointestinal implant device comprises: a flexible, floppy sleeve, open at both ends, to extend into the duodenum; a collapsible anchor coupled to a proximal portion of the sleeve; and a drawstring threaded through a proximal end of the anchor and drawn into the inner tube by the grasper. At least a proximal portion of the anchor may be captured within the retrieval hood such that the retrieval hood covers a plurality of protrusions on a surface of the gastrointestinal implant device, the plurality of protrusions comprising both proximal facing protrusions and distal facing protrusions of the gastrointestinal implant device. The plurality of protrusions may comprise a plurality of muscle-penetrating barbs.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1A is a diagram of a retrieval device and gastrointestinal implant, in accordance with the prior art.

FIG. 1B is a diagram of a retrieval device with a short endoscope hood, along with a collapsed anchor, in accordance with the prior art.

FIG. 2 is a diagram of a short endoscope hood retrieval device with a collapsed anchor, with the anchor refracted into the hood and with distal barbs exposed, in accordance with the prior art.

FIG. 3 is a diagram of a retrieval device with an extended endoscope hood, in accordance with an embodiment of the invention, with a collapsed anchor.

FIG. 4 is a diagram of a retrieval device in accordance with an embodiment of the invention, with a collapsed anchor retracted into the extended hood and with distal barbs fully covered.

FIGS. 5A-5C are schematic diagrams of an atraumatic flared retrieval hood, in accordance with an embodiment of the invention.

FIG. 6 is a diagram illustrating measurement of a bend radius of an atraumatic flared retrieval hood, in accordance with an embodiment of the invention.

### DETAILED DESCRIPTION OF THE INVENTION

A description of example embodiments of the invention follows.

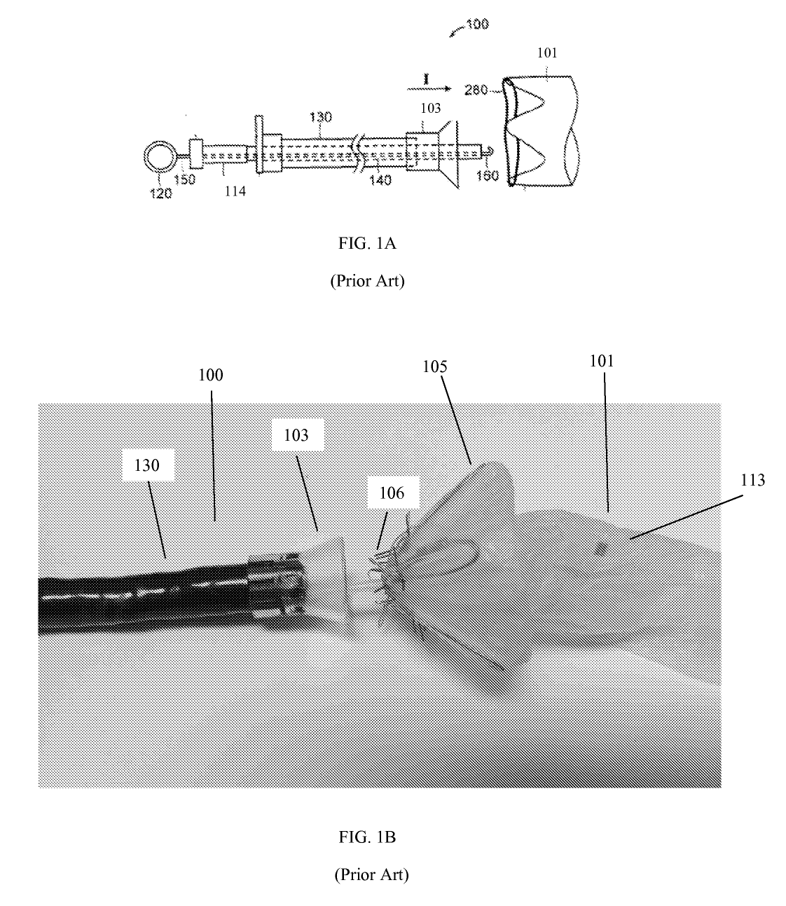

In a previous retrieval system for removing a gastrointestinal implant device from patients, removal of the gastrointestinal implant device has been achieved through an endoscopic system in less than about fifteen minutes. FIG. 1A is a diagram of such a retrieval device 100 that can be used to collapse and remove or reposition a gastrointestinal implant device 101, in accordance with the prior art, as described in U.S. Pat. No. 8,771,219, the entire teachings of which are incorporated by reference. The retrieval device 100 may include a handle 114 supporting an actuator 120. The retrieval device 100 further may include an elongated member 150, such as a wire. The elongated member 150 is slidably disposed within the handle 114. The actuator 120 is adapted to attach to a proximal end of the elongated member 150. The retrieval device 100 further may include an inner tube 140. The inner tube 140 defines a lumen within which the elongated member 150 is slidably disposed. The inner tube 140 is adapted for insertion into a natural bodily lumen through an endoscope working channel or a catheter. The inner tube 140 is fixed to a distal end of the handle 114.

A grasper 160, a hook in this embodiment, is coupled at a distal end of the elongated member 150 and is adapted to grasp a feature of an implantable device 101, such as drawstring 280, such that manipulation of the drawstring 280 can reduce at least one dimension (e.g., the diameter) of the implantable device 101. The grasper 160 may be any means of grasping a drawstring of an implantable device. The grasper 160 attached to a distal portion of the elongated member 150, is disposed within a lumen of the inner tube 140.

The retrieval device 100 may further include an outer tube 130. The outer tube 130 also defines a lumen within which the inner tube 140 may be slidably disposed. In one embodiment, the outer tube 130 is an insertion tube of an endoscope. For example, if the retrieval device 100 is being used within the gastrointestinal tract, the endoscope may be a gastroscope, such as the Olympus GIF Q160, 9.8 mm OD, sold by Olympus Corporation of Tokyo, Japan.

The retrieval device 100 also includes a retrieval hood 103. The retrieval hood 103 may be attached to a distal end of the outer tube 130. The retrieval hood 103 is adapted to capture at least a proximal portion of the implantable device. In some embodiments, the retrieval hood 103 is coupled to the outer tube 130 using an interference fit, where the diameter of the proximal end of the retrieval hood 103 is slightly larger than the distal end of the outer tube 130. In other embodiments, the retrieval hood 103 may be coupled to the outer tube 130 using alternative mechanical, chemical, or bonding techniques.

As shown in FIG. 1A, the grasper 160 coupled to the distal end of the elongated member 150, is advanced towards a drawstring 280 positioned on the proximal end of the implantable device 101 by pushing on the actuator 120 (as indicated by arrow I.) The distal end of the grasper 160 can extend distally beyond the outer tube 130, the retrieval hood 103, and the inner tube 140. The gastrointestinal implant device 101 can comprise a flexible, floppy sleeve (see 113 in FIG. 1B), open at both ends, to extend into the duodenum; a collapsible anchor 105 (see FIG. 1B) coupled to a proximal portion of the sleeve 113; and a drawstring 280 (see FIG. 1A) threaded through a proximal end of the anchor 105 (see FIG. 1A) and drawn into the inner tube 140 by the grasper 160.

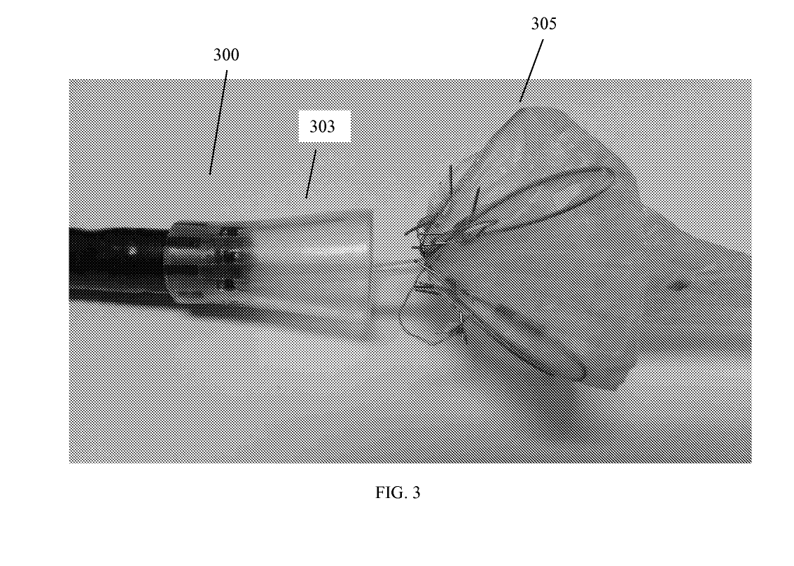

In accordance with an embodiment of the present invention, any of the features of the retrieval device 100 of FIG. 1A, may be used in combination with an extended endoscope hood, such as shown in the embodiments of FIG. 3 and following, in place of the short hood 103 of FIG. 1A.

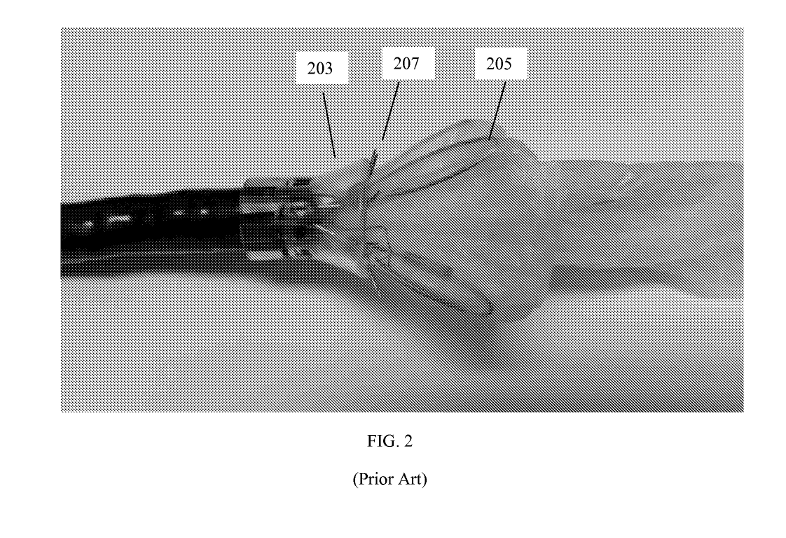

FIG. 1B is a further diagram of a retrieval device 100 with a short endoscope hood 103, collapsing a gastrointestinal implant device 101, in accordance with the prior art. The grasper (see 160 in FIG. 1A) and retrieval hood 103 are used in conjunction with the endoscope 130. Once located, the drawstring (see 280 in FIG. 1A) provided on the implant 101 is grasped with the grasper (see 160 in FIG. 1A) and pulled into the inner tube 140 (see FIG. 1A) to collapse the anchor 105. The drawstrings 280 (see FIG. 1A) are located on the anchor 105 such that pulling on them collapses the anchor 105 radially. This allows the anchor protrusions, such as muscle-penetrating barbs 106, to withdraw from the tissue. Once fully collapsed, the hood 103 on the endoscope 130 is advanced over the barbs 106 to cover them, as shown in FIG. 2, after which the implant 101 is safe to remove from the gastrointestinal tract through the mouth.

However, there is an ongoing need for endoscope devices that increase safety and ease of use. In FIG. 2, it can be seen that in the prior art device, the hood 203 has a comparatively short length, which is about 11 mm as measured from the distal end of the endoscope to the distal end of the hood. Therefore, even with the anchor 205 retracted into the hood 203, the distal muscle-penetrating barbs 207 are exposed, which poses a potential danger to the patient as the implant is removed through the esophagus and mouth.

In response to concerns regarding such potential danger to the patient, among other goals, an embodiment according to the present invention provides an extended endoscope hood, which comprises several features, including a length configured to cover both proximal facing protrusions and distal facing protrusions on a surface of the gastrointestinal implant device. The atraumatic flared retrieval hood comprises a diameter configured to fit within a human esophagus without damage to the esophagus, and comprises a material of a bend radius configured to move slidably against the esophagus without damage to the esophagus.

FIG. 3 is a diagram of a retrieval device 300 in accordance with an embodiment of the invention, with a collapsed anchor 305. It can be seen that the hood 303 is of a longer length and shallower angle than the prior art hood 103 of FIG. 1B. By comparison, the prior art hood of FIG. 1B is short and only covers the proximal barbs 106 of the anchor 105, thereby limiting the implant to movement in a single direction during device retrieval. The prior art hood of FIG. 1B was made comparatively short in order to provide a maximum field of view and maneuverability for the endoscope.

By contrast, in accordance with an embodiment of the present invention, the hood 303 is designed to increase safety and ease of use. The hood 303 covers both proximal and distal barbs, thereby preventing any scratching or tearing in the upper gastrointestinal tract during removal of the gastrointestinal implant device. It was previously thought that hood length would affect field of view and scope maneuverability negatively; however, it has been determined that the safety and ease of use of a hood in accordance with an embodiment of the invention are benefits that outweigh the slight differences in view and control.

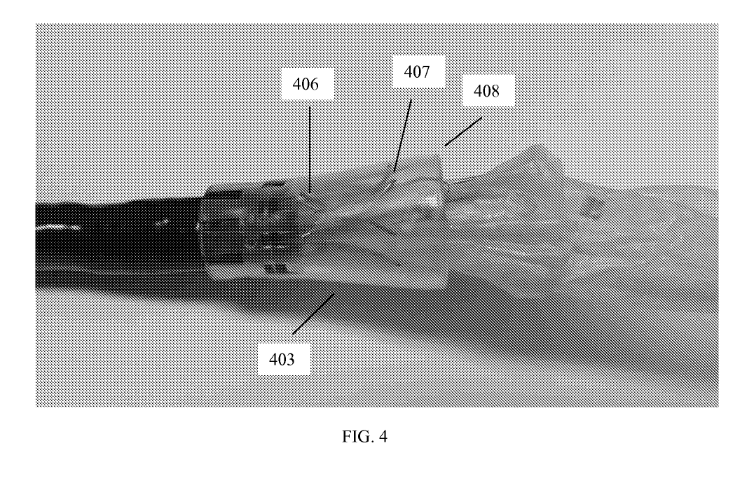

FIG. 4 is a diagram of an extended endoscope hood device in accordance with an embodiment of the invention, with a collapsed anchor retracted into the hood. Both the proximal barbs 406 and distal barbs 407 are fully covered by the hood 403. With reference to FIG. 3, in use, the grasper (see 160 in FIG. 1A) is used to collapse the implant anchor 305 away from the duodenal wall. Once the anchor 305 is sufficiently collapsed and pulled away from the wall, it is retracted into the retrieval hood 303. In accordance with an embodiment of the invention, the geometry and material properties of the hood 303 allow for safe navigation down the upper gastrointestinal tract and into the pylorus and back. They also provide adequate field of view and range of motion. As shown in FIG. 4, when the anchor is fully collapsed and retracted against the endoscope face, the hood 403 fully covers both proximal barbs 406 and distal barbs 407. With all barbs covered, the user is able to move the endoscope in all directions without causing damage. The wall geometry of the hood 403 allows the barbs 406, 407 to catch the distal edge 408 of the hood, so that the barbs 406,407 are folded over to be safely covered by the hood 403.

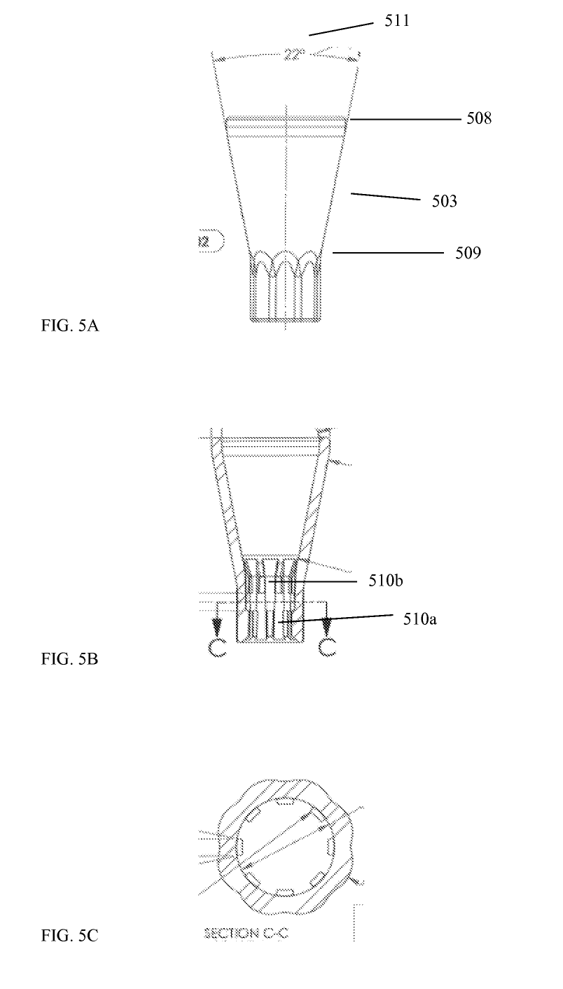

FIGS. 5A-5C are schematic diagrams of an atraumatic flared retrieval hood, in accordance with an embodiment of the invention. FIG. 5A is a side view; FIG. 5B is a vertical section view; and FIG. 5C is a sectional view through a base of the hood, in accordance with an embodiment of the invention. The atraumatic flared retrieval hood 503 (see FIG. 5A) is configured to attach to and fit over a distal end of the endoscope. The length of the atraumatic flared retrieval hood 503 is between about 18 mm and about 36 mm from the distal end of the endoscope (positioned at approximately location 509) to the distal end 508 of the atraumatic flared retrieval hood 503. This length allows the atraumatic flared retrieval hood 503 to cover both proximal and distal facing barbs (406, 407 of FIG. 4) of the gastrointestinal implant device, although a length that is too long could make the hood 503 difficult to maneuver. In one embodiment, the overall length of the hood is 1.39 inches (35.3 mm), such as between about 29.2 mm and about 47.2 mm, and its effective length (from the distal end of the endoscope to the distal end 508 of the atraumatic flared retrieval hood 503) is about 0.947 inches (24.1 mm), such as between about 18 mm and about 36 mm.

In accordance with an embodiment of the invention, the largest diameter (for example at the distal end 508) of the atraumatic flared retrieval hood 503 may be between about 10 mm and about 20 mm, with the high end of the range being approximately the largest diameter that can fit through the human esophagus, and the low end of the range being determined by the diameter of the endoscope. An angle 511 formed by the walls of the atraumatic flared retrieval hood 503 may be between about 20 degrees and 30 degrees, such as about 22 degrees. Although not shown in FIGS. 5A-5C, the distal end 508 of the atraumatic flared retrieval hood 503 may include a bead feature around the diameter of the distal end 508. The atraumatic flared retrieval hood 503 also may include retention features 510a/510b, shown in vertical sectional view in FIG. 5B. The retention features 510a/510b may include openings between neighboring features around a circumference of the hood 503, between each set of features 510a and 510b, that assist in fitting the hood 503 onto the endoscope. A bottom row of retention features 510a (see FIG. 5B) prevents motion of the retrieval hood 503 on the endoscope in a distal direction, whereas a top row of retention features 510b prevents motion of the retrieval hood 503 on the endoscope in a proximal direction. The features 510a and 510b may be offset from one another, so that openings between features 510a are offset from openings between features 510b. Such an offset improves moldability of the hood 503. The endoscope may, for example, be a gastroscope, such as the Olympus GIF Q160, 9.8 mm OD, sold by Olympus Corporation of Tokyo, Japan; or another endoscope with a diameter from 8 mm to 13.2 mm.

Generally, the esophagus is not necessarily cylindrical in shape, and is actively moving, so that it can tend to flatten any retrieval hood as it is passed through the esophagus. Applicants have realized that a retrieval hood that does not have a sufficiently large bend radius could potentially form a cutting edge when flattened, which can pinch or tear the tissue of the esophagus. Therefore, in accordance with an embodiment of the invention, the atraumatic flared retrieval hood 503 has a bend radius that is sufficiently large that the hood does not pinch or tear tissue when it is folded, which may be of particular concern for a patient's esophagus and/or epiglottis as a gastrointestinal implant device is removed. In order to prevent pinching or tearing, the bend radius must be larger than a minimum dimension, which may, for example, be at least about 0.055 inches (1.40 mm), or at least about 0.060 inches (1.52 mm). With such a minimum bend radius, the hood 503 can be almost fully collapsed at its distal diameter without creating sharp or traumatic edges. The hood may, for example, have a thickness of about 0.072 inches (1.83 mm), although a thickness such as about 0.053 inches (1.35 mm) to about 0.078 inches (1.98 mm) may be used.

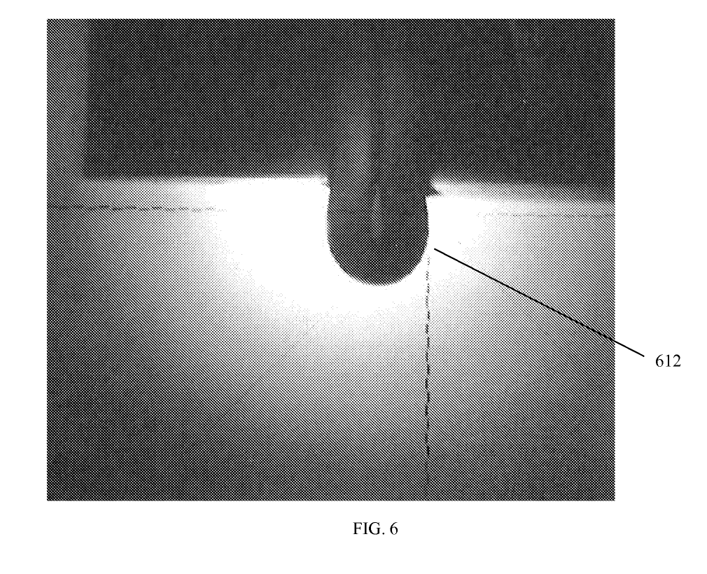

FIG. 6 is a diagram illustrating measurement of a bend radius of an atraumatic flared retrieval hood, in accordance with an embodiment of the invention. The bend radius is measured at a bend 612 of the atraumatic flared retrieval hood when the atraumatic flared retrieval hood is in a flattened configuration. The bend radius may be measured, for example, using a 1-inch clamp sold by Instron® of Norwood, Mass., U.S.A., using a three-point diameter function that calculates a bend radius using an optical comparator.

In accordance with an embodiment of the invention, the atraumatic flared retrieval hood 503 has a durometer that is sufficient to prevent the barbs from penetrating through the hood. The durometer must also be high enough that the material does not stretch, causing a sharp edge, and soft enough that the hood can deform to fit through the esophagus. For example, the atraumatic flared retrieval hood may comprise a material of a durometer between about Shore 70A and about Shore 95A. The atraumatic flared retrieval hood 503 may, for example, comprise at least one of a urethane, an elastomer, and a silicone; such as a transparent urethane. The urethane Tecoflex™, sold by The Lubrizol Corporation of Wickliffe, Ohio, U.S.A., is an example of a material that may be used. In one example, the material is a thermoplastic urethane with a durometer of Shore 85A. The compressive force (flattening force) of the material may be around 1 lbf +/−0.25 lbf.

It will be appreciated that retrieval devices taught herein can be used to remove a gastrointestinal implant device from a gastrointestinal tract, and/or to reposition a gastrointestinal implant device within a gastrointestinal tract.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. An endoscope hood device for moving a gastrointestinal implant device within a natural lumen of a gastrointestinal tract, the endoscope hood device comprising:
a collapsible, atraumatic flared retrieval hood configured to attach to and fit over a distal end of an endoscope;
the atraumatic flared retrieval hood comprising a length of between about 18 mm and about 36 mm from the distal end of the endoscope to a distal end of the atraumatic flared retrieval hood;
the atraumatic flared retrieval hood comprising a largest diameter of between about 10 mm and about 20 mm;
the atraumatic flared retrieval hood comprising a material of a durometer between about Shore 70A and about Shore 95A; and
the atraumatic flared retrieval hood comprising a material of a bend radius of at least about 0.055 inches, the bend radius being measured at a bend of the atraumatic flared retrieval hood when the atraumatic flared retrieval hood is in a flattened configuration.

2. The endoscope hood device of claim 1, wherein the bend radius of the atraumatic flared retrieval hood is at least about 0.060 inches.

3. The endoscope hood device of claim 1, wherein the bend radius is measured at a distal tip of the atraumatic flared retrieval hood.

4. The endoscope hood device of claim 1, wherein the atraumatic flared retrieval hood comprises at least one of a urethane, an elastomer, and a silicone.

5. The endoscope hood device of claim 1, wherein the atraumatic flared retrieval hood comprises a transparent urethane.

6. A retrieval device comprising an endoscope hood device for moving a gastrointestinal implant device within a natural lumen of a gastrointestinal tract, the endoscope hood device comprising:
(i) a collapsible, atraumatic flared retrieval hood configured to attach to and fit over a distal end of an endoscope;
(ii) the atraumatic flared retrieval hood comprising a length of between about 18 mm and about 36 mm from the distal end of the endoscope to a distal end of the atraumatic flared retrieval hood;
(iii) the atraumatic flared retrieval hood comprising a largest diameter of between about 10 mm and about 20 mm;
(iv) the atraumatic flared retrieval hood comprising a material of a durometer between about Shore 70A and about Shore 95A; and
(v) the atraumatic flared retrieval hood comprising a material of a bend radius of at least about 0.055 inches, the bend radius being measured at a bend of the atraumatic flared retrieval hood when the atraumatic flared retrieval hood is in a flattened configuration;
the retrieval device further comprising the endoscope, the endoscope defining a lumen and providing an outer tube of the retrieval device.

7. The retrieval device of claim 6, further comprising an inner tube defining a lumen and adapted for insertion into the endoscope;
an elongated member having a proximal end and a distal end, the elongated member slidably disposed within the lumen of the inner tube;
a grasper disposed at the distal end of the elongated member, adapted to engage the gastrointestinal implant device to collapse at least a portion of the gastrointestinal implant device when operated.

8. The retrieval device of claim 6, further comprising the gastrointestinal implant device, wherein the gastrointestinal implant device comprises:
a flexible, floppy sleeve, open at both ends, to extend into the duodenum;
a collapsible anchor coupled to a proximal portion of the sleeve; and
a drawstring threaded through a proximal end of the anchor and drawn into the inner tube by the grasper;
at least a proximal portion of the anchor being captured within the retrieval hood such that the retrieval hood covers a plurality of protrusions on a surface of the gastrointestinal implant device, the plurality of protrusions comprising both proximal facing protrusions and distal facing protrusions of the gastrointestinal implant device.

9. The retrieval device of claim 6, wherein the plurality of protrusions comprise a plurality of muscle-penetrating barbs.
